(19) United States
(12) Patent Application Publication
(10) Pub. No.: US 2019/0008454 A1
(43) Pub. Date: Jan. 10, 2019

(54) SMART CLOTHING

(71) Applicant: Sabrina Fashion Industrial Corp., New Taipei City (TW)

(72) Inventors: Yu-Ming Hsu, New Taipei City (TW); Shang-Fu Chiang, New Taipei City (TW); Wei-Han Hsiao, New Taipei City (TW)

(73) Assignee: Sabrina Fashion Industrial Corp., New Taipei City (TW)

(21) Appl. No.: 15/798,408

(22) Filed: Oct. 31, 2017

Related U.S. Application Data
(60) Provisional application No. 62/529,434, filed on Jul. 6, 2017.

(51) Int. Cl. A61B 5/00; A41D 1/00; A61B 5/0408

(52) U.S. Cl. CPC A61B 5/6843; A41D 1/002; A61B 2562/0209; A61B 5/6804; A61B 5/04085

(57) ABSTRACT

A smart clothing is provided, including a main body, a detecting area, and a compression area. The detecting area is disposed on the main body. The compression area is disposed on the main body opposite to the detecting area, and the compression area has at least one highest point. The position of the highest point is higher than the position of the detecting area. The compression area extends from the highest point to two sides of a wearer's body.

### CROSS-REFERENCE TO RELATED APPLICATION

This application claims the priority benefits of U.S. provisional application Ser. No. 62/529,434, filed on Jul. 6, 2017. The entirety of the above-mentioned patent application is hereby incorporated by reference herein and made a part of this specification.

### BACKGROUND OF THE INVENTION

### 1. Field of the Invention

The invention is related to a smart clothing, and more particularly, to a smart clothing capable of maintaining wearing comfort and detection stability simultaneously.

### 2. Description of Related Art

In prior art, the smart clothing is usually sized smaller than general clothing of the same size, so as to obtain better detection signal. No matter when the wearer rests or exercises, the smaller-sized smart clothing keeps the detecting area stably attaching to wearer's body surface. However, the smaller-sized smart clothing causes chest pressure, which leads to breathing difficulties, so the athletic performance may be poor. Besides, it is difficult to put on and take off smaller-sized smart clothing, and the wearing comfort is also affected, so the consumer's purchase intention is lowered. On the hand, if the wearer wears loose-fitting clothes, the detecting area is not able to stably attach to wearer's body surface, so the wearer's movement causes unexpected shaking of detecting area and the signal quality is seriously affected. Moreover, the wearer's chest expands and contracts continuously with inhalation and exhalation of each breath, which causes unexpected displacement of chest detecting area, so the signal detection is affected.

Based on the above, a smart clothing capable of stably attaching to wearer's body and improving wearing comfort is urgently needed.

### SUMMARY OF THE INVENTION

The invention provides a smart clothing capable of stably attaching to wearer's body for detection stability and providing wearing comfort simultaneously.

The invention provides a smart clothing including a main body, a detecting area, and a compression area. The detecting area is disposed on the main body. The compression area is disposed on the main body opposite to the detecting area, and the compression area has at least one highest point. The position of the highest point is higher than the position of the detecting area. The compression area extends from the highest point to two sides of a wearer's body.

Based on the above, the invention provides a smart clothing including a detecting area and a compression area, wherein the position of the highest point of the compression area is higher than the position of the detecting area. Therefore, the tightening force of the compression area is not directly exerted on wearer's chest, so breathing difficulties can be prevented and better wearing comfort can be achieved. In addition, the compression area is located on the back side of the main body, so the tightening force can be applied following wearer's respiratory movements and along the scapula towards the highest point of the compression area. As a result, the detecting area is stably attached to the wearer's body, so unexpected displacement of the detecting area can be prevented and signal quality is effectively enhanced without affecting normal breathing.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic front view of a smart clothing according to an embodiment of the invention.

FIG. 2 is a schematic back view of a smart clothing according to the first embodiment of the invention.

FIG. 3 is a schematic view of a smart clothing of the invention when worn by the wearer.

FIG. 4 is a schematic back view of a smart clothing according to the second embodiment of the invention.

FIG. 5 is a schematic back view of a smart clothing according to the third embodiment of the invention.

FIG. 6 is a schematic back view of a smart clothing according to the fourth embodiment of the invention.

FIG. 7 is a schematic back view of a smart clothing according to the fifth embodiment of the invention.

FIG. 8 is a schematic back view of a smart clothing according to the sixth embodiment of the invention.

FIG. 9 is a schematic back view of a smart clothing according to the seventh embodiment of the invention.

FIG. 10 is a curve diagram of the wearer's heart rate over time when wearing loose-fitting clothing during exercise.

FIG. 11 is a curve diagram of the wearer's heart rate over time when wearing the smart clothing of the invention during exercise.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

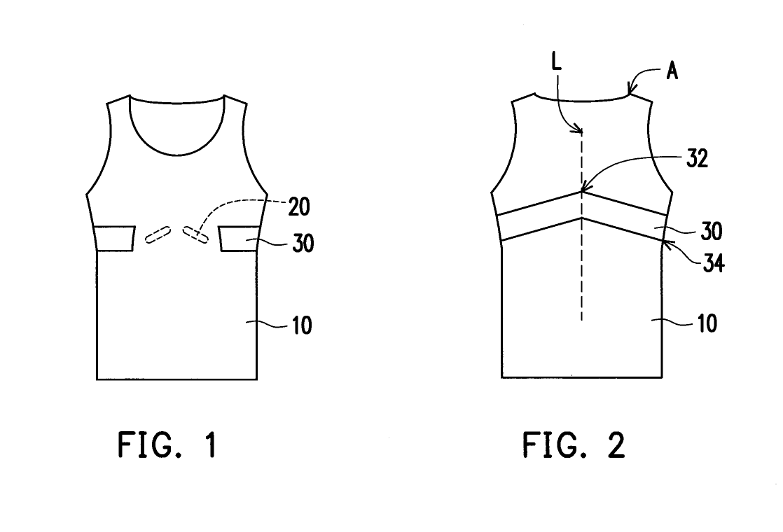

First, referring to FIG. 1 and FIG. 2, in the present embodiment, a smart clothing includes a main body 10, a detecting area 20, and a compression area 30. The detecting area 20 shown in FIG. 1 is disposed on the front inner side of the main body 10, and the compression area 30 having at least one highest point 32 shown in FIG. 2 is disposed on the back side of the main body 10 opposite to the detecting area 20. More particularly, the position of the highest point 32 is higher than the position of the detecting area 20, so as to provide wearing comfort and detection stability at the same time. The distance between the position of the highest point 32 and the position of the detecting area 20 may be 10 cm to 20 cm, for example. In the present embodiment, two sides of the compression area 30 can further extend to two sides of the detecting area 20 for better tightening force, but the invention is not limited thereto, and tightening force of the compression area 30 can also be effectively exerted for detection stability on the condition that only the compression area 30 having at least one highest point 32 is disposed on the back side of the main body 10 (as shown in FIG. 2).

In the present embodiment, the tensile strength of the compression area 30 is higher than the tensile strength of the main body 10, and the tensile strength of the detecting area 20 is higher than the tensile strength of the compression area 30. Besides, the compression area 30 has a stretching ability in the extension direction thereof.

Therefore, the compression area 30 creates a tightening force relative to the main body 10, so the detecting area 20 can be stably attached to the wearer's body. The detecting area 20 has the highest tensile strength, so shape changing or unexpected displacement can be prevented even when an outside force is exerted. More particularly, the material of the main body 10 and the detecting area 20 may be 84% nylon and 16% spandex, and the material of the compression area 30 may be 76% nylon and 24% spandex. Besides, the material of the compression area 30 can also include elastic band, elastic webbing, or elastic yarn. Otherwise, the material of the main body 10, the detecting area 20, and the compression area 30 may all be 84% nylon and 16% spandex, and the post-processing method may be adopted to make the tensile strength of the compression area 30 higher than the tensile strength of the main body 10. The post-processing method may include stitching multiple elastic treads into the smart clothing to form the compression area 30, or other methods such as smocking. However, the invention is not limited thereto, and the other materials can be used as long as the tensile strength of the compression area 30 is higher than the tensile strength of the main body 10, and the tensile strength of the detecting area 20 is higher than the tensile strength of the compression area 30.

In the present embodiment, the compression area 30 may be stitched onto the main body 10, for example, and other clothing processing methods (such as heat pressing, adhesion or patchwork) can also be adopted to combine the compression area 30 to the main body 10. Due to the processing method of the compression area 30, there may be processing mark on the outer surface or the inner surface of the main body 10.

In the present embodiment, the detecting area 20 has a plurality of electrodes, and can be disposed adjacent to the xiphoid process of wearer's chest. For example, the electrodes may be the conductive ink formed by printing, and the other clothing methods (such as stitching, heat pressing, adhesion or patchwork) may be adopted to combine the conductive materials (such as conductive yarn or conductive cloth) to the main body 10 so as to form the detecting area 20. The shape of the electrodes may include long strip, circle or other shapes. The arrangement direction of the electrodes is preferably the same as the extension direction of the compression area 30 on the back side of the wearer. More particularly, the arrangement direction of the electrodes is preferably the same as the extension direction of the wearer's ribs, but the invention is not limited thereto. The detecting area 20 is electrically connected to an ECG device (not shown). For example, the ECG device may electrically connected to the detecting area 20 through fasteners (buttons) on the front side of the main body 10, so that the ECG device is able to store or transfer the signal detected by the detecting area 20 to the external electric device. Besides, the ECG device can be disposed on the other positions of the main body 10 according to user's requirements, such as collar area, armpit, shoulder or hem, or the ECG device can be directly integrated into the main body 10 and electrically connected to the detecting area 20 through internal routing.

Referring to FIG. 2, the shape of the compression area 30 may be an inverted V shape on the back side of the wearer, but the invention is not limited thereto, and the compression area 30 may have other shapes or designs as long as there is at least one highest point on the back side of the main body 10. In the present embodiment, the compression area 30 extends from the highest point 32 along the scapula to two sides of the wearer's body. More particularly, the highest point 32 may be preferably located at the central line L on the back side of the main body 10, and the compression area 30 may symmetrically extend from the highest point 32 to two sides of the wearer's body with respect to the central line L as the symmetric center. However, the invention is not limited thereto, and the highest point 32 may be slightly deviated from the central line L as long as it is still located in the central area on the back side of the wearer, so as to concentrate the force, which helps the detecting area 20 closely attach to the wearer's body.

Referring to FIG. 2, the compression area 30 also has at least one lowest point 34, and the ratio of the distance between the high shoulder point A of the main body 10 and the highest point 32 to the distance between the highest point 32 and the lowest point 34 may be 1:5 to 5:1, for example. In the present embodiment, the highest point 32 may be located at the highest point of wearer's thoracic vertebra, and the lowest point 34 may be located at the lowest point of wearer's thoracic vertebra. The length of the main body 10 may be 62 cm, for example. The width of the compression area 30 may be 2 cm to 15 cm, for example, so as to provide sufficient tightening force.

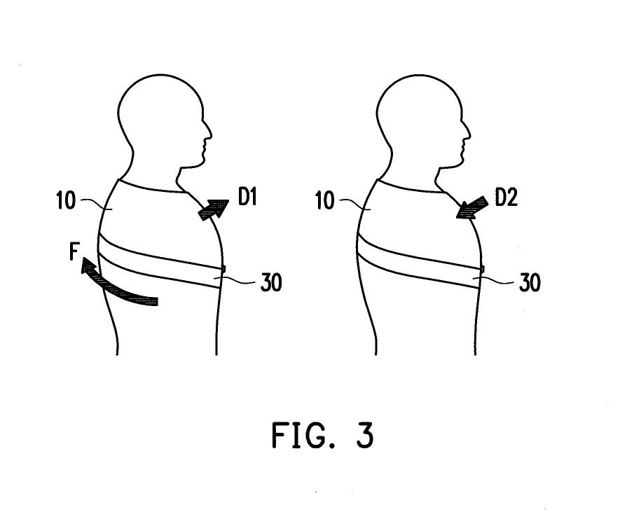

Referring to FIG. 3, the wearer's chest expands and contracts continuously with inhalation and exhalation of each breath, and the direction D1 indicates the chest movement direction during inhalation and the direction D2 indicates the chest movement direction during exhalation. The position of the highest point of compression area 30 on the back side of the main body 10 is higher than the position of the detecting area, so a tightening force F having an obliquely upward direction is created and applied following the wearer's respiratory movements. Therefore, the tightening force F of the compression area 30 is not directly exerted on wearer's chest, so breathing difficulties can be prevented and better wearing comfort can be achieved. In addition, the detecting area can be stably attached to the wearer's body without affecting normal breathing or wearing comfort, so unexpected displacement or shaking of the detecting area can be prevented and signal quality is effectively enhanced.

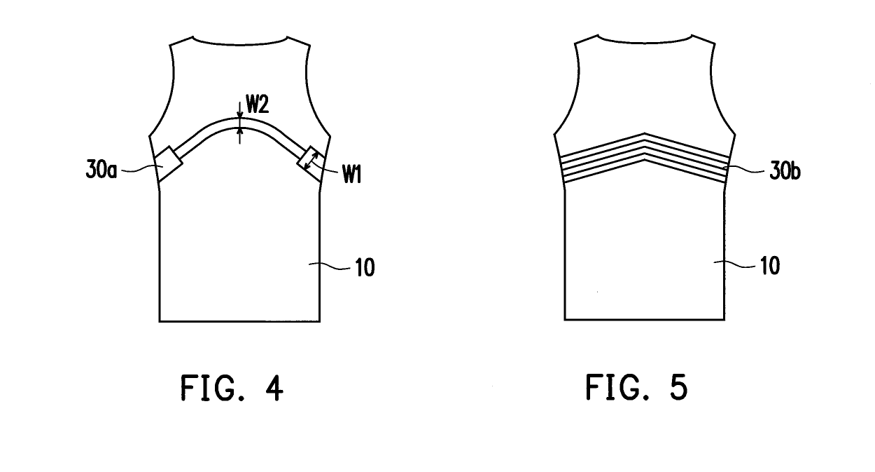

FIG. 4 to FIG. 9 are mainly used to explain the different shapes or designs of compression area on the back side of the main body, and the configuration of the front side of the main body may be all the same as shown in FIG. 1. The embodiments shown in FIG. 4 to FIG. 9 are similar to the embodiment shown in FIG. 2, and therefore the same components are represented by the same reference numerals and are not repeated herein.

Referring to FIG. 4, the compression area 30a may have at least two different widths W1 and W2, and the width W1 near two sides of the wearer's body is larger than the width W2 of the central area on the back side of the wearer, for example. However, even though there are only two different widths W1 and W2 shown in FIG. 4, the number of different widths in the compression area are only exemplary, and the invention is not limited thereto. In other words, the number of different widths in the compression area can be adjusted according to requirements, as long as the width near two sides of the wearer's body is larger than the width of the central area on the back side of the wearer, so that the detecting area can be attached to the wearer more smoothly. Besides, comparing to the compression area having consistent overall width (the compression area 30 of FIG. 2, for example), the compression area 30a having smaller width of the central area provides lower tightening force, so wearing comfort is further improved.

Referring to FIG. 5, the compression area 30b may be composed of a plurality of compression elements. More particularly, the compression elements may be arranged together side by side, and there can be gap between each of the compression elements. The compression area 30b has the stretching ability in the extension direction thereof.

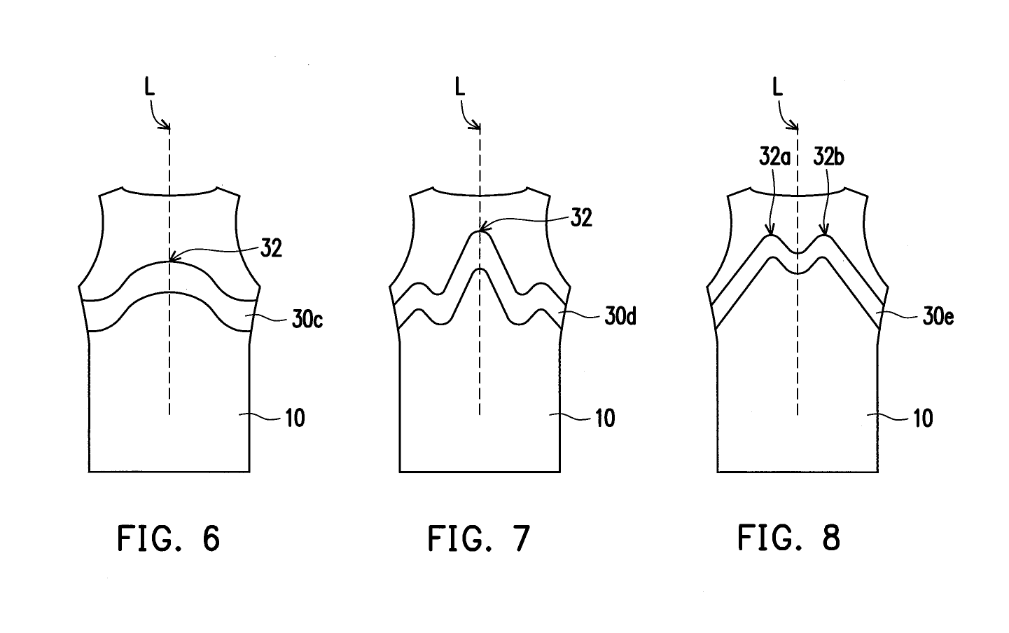

Referring to FIG. 6, FIG. 7 and FIG. 8, the compression area 30c, 30d and 30e may have different shapes or designs as long as there is at least one highest point on the back side of the main body 10. Except for the inverted V shape of compression area 30 shown in FIG. 2, the compression area 30c, 30d and 30e shown in FIG. 6, FIG. 7 and FIG. 8 can be wavy designs. The compression area 30c and 30d shown in FIG. 6 and FIG. 7 have one highest point 32 respectively, and the compression area 30e shown in FIG. 8 has two highest points 32a and 32b. However, even though there is only one highest point 32 shown in FIG. 6 and FIG. 7 and only two highest points 32a and 32b shown in FIG. 8, the number of highest points in the compression area are only exemplary, and the invention is not limited thereto, and the number of highest points can be adjusted according to requirements as long as the highest points are still located in the central area on the back side of the wearer.

Referring to FIG. 6 and FIG. 7, the highest point 32 may be preferably located at the central line L on the back side of the main body 10, and the compression area 30c and 30d may symmetrically extend from the highest point 32 to two sides of the wearer's body with respect to the central line L as the symmetric center. Referring to FIG. 8, the highest points 32a and 32b may be symmetrically located at two sides of the central line L on the back side of the main body 10, and the distance between the central line L and each of the highest points 32a and 32b may be the same. The compression area 30e may symmetrically extend from the highest points 32a and 32b to two sides of the wearer's body with respect to the central line L as the symmetric center. However, the invention is not limited thereto, and the highest point 32 may be slightly deviated from the central line L as long as it is still located in the central area on the back side of the wearer, the distance between the central line L and each of the highest points 32a and 32b may be slightly different. The compression area is able to provide tightening force corresponding at two sides of the detecting area 20, which helps the detecting area 20 closely attach to the wearer's body without causing chest pressure or breathing difficulties.

The compression area may be composed of composite material, so as to provide appropriate tightening force according to requirements. In other words, the compression area may be composed of at least two different kinds of materials, and the different materials can be arranged side by side or in lamination, for example. As a result, the compression area may have at least two different tensile strengths, and the tensile strength near two sides of the wearer's body is larger than the tensile strength of the central area on the back side of the wearer.

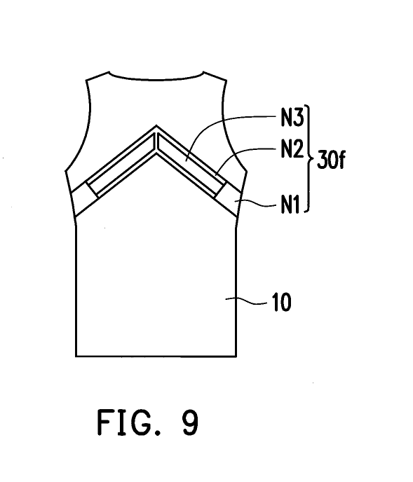

Referring to FIG. 9, for example, the compression area 30f is composed of a first area N1, a second area N2, and a third area N3. The tensile strength of the main body 10, the first area N1, the second area N2, and the third area N3 can be arranged in the following listed order: the main body 10 <the third area N3 <the second area N2 <the first area N1, the main body 10 <the second area N2 <the third area N3 <the first area N1, or the main body 10 <the second area N2=the third area N3 <the first area N1. However, even though there are only three different kinds of materials (the first area N1, the second area N2, and the third area N3) shown in FIG. 9, the number of different materials in the compression area are only exemplary, and the invention is not limited thereto. In other words, the number of different materials in the compression area can be adjusted according to requirements, as long as the tensile strength near two sides of the wearer's body is larger than the tensile strength of the central area on the back side of the wearer.

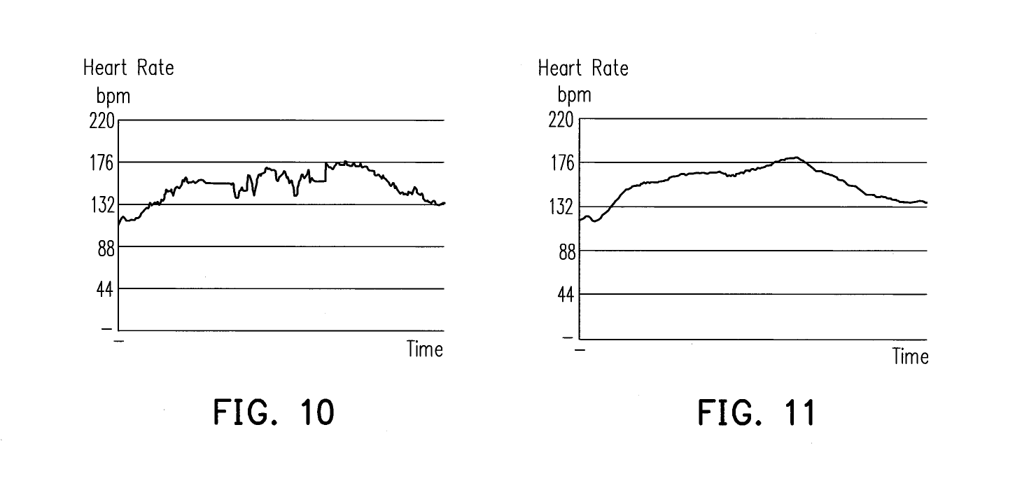

Referring to FIG. 10, when the wearer wears loose-fitting clothing during exercise, the detecting area is not able to attach to wear's body, so the detecting area shakes due to wearer's movements and signal quality is seriously affected. Referring to FIG. 11, when the wearer wears the smart clothing of the invention during exercise, the detecting area is able to stably attach to wear's body through compression area, so better signal quality can be obtained and smooth curve is shown in FIG. 11.

To prove that the smart clothing provided in the invention is able to prevent breathing difficulties and chest pressure, the following experimental data is provided. The smart clothing of the invention and the commercially available traditional smart clothing are put on test dummies, and then the pressure value of detecting area are evaluated. The bust of the test dummy is 101 cm, and the busts of the smart clothing of the invention and the commercially available traditional smart clothing are both 76 cm. The pressure value of detecting area of the smart clothing of the invention is 9 g/cm², and the pressure value of detecting area of commercially available traditional smart clothing is 34 g/cm². Therefore, it is clear that the pressure value of detecting area of commercially available traditional smart clothing is much higher than the pressure value of detecting area of the smart clothing of the invention, so the smart clothing of the invention is able to prevent breathing difficulties and chest pressure and provide better wearing comfort.

Based on the above, the invention provides a smart clothing including a detecting area and a compression area having a highest point, wherein the position of the highest point of the compression area is higher than the position of the detecting area. Therefore, the tightening force of the compression area is not directly exerted on wearer's chest, so the wearer can breathe smoothly and wearing comfort can be effectively enhanced. In addition, the compression area is located on the back side of the main body, so the tightening force can be applied following wearer's respiratory movements and along the scapula towards the highest point of the compression area. As a result, the detecting area is stably attached to the wearer's body, so unexpected displacement or shaking of the detecting area can be prevented and signal quality is effectively enhanced without affecting normal breathing, and the pressure value of detecting area of the smart clothing of the invention is much lower than the pressure value of detecting area of commercially available traditional smart clothing.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A smart clothing, comprising:
a main body;
a detecting area disposed on the main body; and
a compression area disposed on the main body opposite to the detecting area, wherein the compression area has at least one highest point, and a position of the highest point is higher than a position of the detecting area, wherein the compression area extends from the highest point to two sides of a wearer's body.

2. The smart clothing of claim 1, wherein the compression area has a stretching ability in an extension direction thereof.

3. The smart clothing of claim 1, wherein the highest point is located at a central line on a back side of the main body, and the compression area symmetrically extends from the highest point to two sides of the wearer's body with respect to the central line as a symmetric center.

4. The smart clothing of claim 1, wherein two sides of the compression area extends to two sides of the detecting area.

5. The smart clothing of claim 1, wherein a distance between the position of the highest point and the position of the detecting area is 10 cm to 20 cm.

6. The smart clothing of claim 1, wherein a width of the compression area is 2 cm to 15 cm.

7. The smart clothing of claim 1, wherein the compression area has at least two different widths, and a width near two sides of the wearer's body is larger than a width of a central area on a back side of the wearer.

8. The smart clothing of claim 1, wherein the compression area has at least two different tensile strengths, and a tensile strength near two sides of the wearer's body is larger than a tensile strength of a central area on a back side of the wearer.

9. The smart clothing of claim 1, wherein the compression area has at least one lowest point, and a ratio of a distance between a high shoulder point of the main body and the highest point to a distance between the highest point and the lowest point is 1:5 to 5:1.

10. The smart clothing of claim 1, wherein a tensile strength of the compression area is higher than a tensile strength of the main body, and a tensile strength of the detecting area is higher than the tensile strength of the compression area.

11. The smart clothing of claim 1, wherein a shape of the compression area is an inverted V shape on a back side of the wearer.

12. The smart clothing of claim 1, wherein the detecting area has a plurality of electrodes, and an arrangement direction of the electrodes is the same as an extension direction of the compression area on a back side of the wearer.

13. The smart clothing of claim 1, wherein the detecting area is electrically connected to an ECG device.
